(19) United States
(12) Patent Application Publication
(10) Pub. No.: US 2019/0297874 A1
(43) Pub. Date: Oct. 3, 2019

(54) METHOD FOR ASSESSING CORNEAL TISSUE QUALITY AND ENDOTHELIAL CELL DENSITY AND MORPHOLOGY

(71) Applicant: CorneaGen Inc., Seattle, WA (US)

(72) Inventors: Adam M. Fox, Newport Beach, CA (US); Caroline Hoover, Seattle, WA (US)

(21) Appl. No.: 16/307,886

(22) PCT Filed: Jun. 5, 2017
(86) PCT No.: PCT/US2017/035920

§ 371 Date: Dec. 6, 2018

Related U.S. Application Data
(60) Provisional application No. 62/347,034, filed on Jun. 7, 2016.

(51) Int. Cl. A01N 1/02; C12N 5/079

(52) U.S. Cl. CPC A01N 1/0221; A01N 1/0252; C12N 5/0621

(57) ABSTRACT

Provided are surprisingly effective methods for assessing corneal tissue quality, comprising: incubating corneal tissue in hypothermic corneal storage media to a temperature above ambient temperature; and assessing, using suitable microscopic examination, endothelial cell density and/or morphology and/or loss. The methods may additionally comprise; storing the corneal tissue in the hypothermic storage media at a temperature in the range of 2-8° C. prior to assessing the corneal tissue above ambient temperature. The methods may involve initially assessing corneal tissue quality at ambient temperature prior to assessing at a temperature above ambient temperature, with intermediate storage at 2-8° C.

### CROSS-REFERENCE TO RELATED APPLICATIONS

This application claims the benefit of priority to U.S. Provisional Patent Application No. 62/347,034, filed 7 Jun. 2016 and entitled “METHOD FOR ASSESSING CORNEAL TISSUE QUALITY AND ENDOTHELIAL CELL DENSITY AND MORPHOLOGY”

### FIELD OF THE INVENTION

Aspects of the present invention relate generally to methods for assessing corneal tissue quality, and more particularly to faster and more accurate methods for assessing measures of corneal tissue quality, including endothelial cell density and/or morphology as indicators of corneal tissue quality for purposes of improving outcomes with respect to corneal tissue storage and/or transplantation.

### BACKGROUND

Effective corneal transplantation from harvested corneal tissue is dependent upon obtaining a good quality assessment of the harvested corneal tissue prior to transplantation. Such quality assessment is often performed multiple times prior to transplantation of the corneal tissue into a recipient. For example, harvested and stored corneal tissue may be quality assessed prior to and/or removal from storage (e.g., hypothermic storage); and prior to and/or after cutting/trimming the corneal tissue for release for transplantation into a recipient.

Typically, corneal tissue is harvested with a rim of sclera (a corneoscleral disc) and placed in corneal storage media. For hypothermic storage, a suitable chondroitin-sulphate-based commercial hypothermic corneal storage media or other suitable media is typically used, examples of which include, but are not limited to: Optisol™ (a corneal storage media sold by Bausch & Lomb, Rochester, N.Y., comprising chondroitin sulfate, dextran 40, Optisol™ base powder, sodium bicarbonate, gentamycin and/or streptomycin sulfate, amino acids, sodium pyruvate, L-glutamate (or L-glutamine), 2-mercaptoethanol, and water); Optisol™ GS; Dexsol; Life 4C; McCarey-Kaufman (M.K., comprising Dextran 40, MK base powder, HEPES buffer, sodium bicarbonate, phenol red, gentamycin and water); Eusol-C; etc. Quality assessment of post-mortem corneal tissue is typically performed as soon as possible. The tissue is removed from the refrigerator and allowed to equilibrate at ambient temperature (room temperature; e.g., 20° C.) for at least one, and typically several hours (e.g., 3 hours), to all day, before performing tissue quality assessment, also at ambient temperature (e.g., in a perfusion specular microscope, such as: Hai Lab; Konan Kerato analyzer; EKA-98; Keeler Konan; etc.). It is long recognized in the art that even warming of corneas above room temperature has a deleterious effect on cell morphology and that folds induced by swelling of corneal tissue at such elevated temperatures results in endothelial cell damage and some cell loss (e.g., see Rootman, et al., Br. J. Opthalmol. 72:545-549, 1988, concluding that incubation of corneal tissue at 37° C. from (1 to 6 hours) in M.K. medium resulted in progressive photo quality loss and reduced cell density, as judged by specular microscopy and wet mount preparations, and indicating that methods of storage which result in greater swelling of stored tissue (such as increased temperature) induce more damage/disruption to endothelial cells. Additionally, incubation of corneal cells above room temperature is contra-indicated in the art for fear of media exhaustion in view of increased tissue metabolism.

Not surprisingly, commercial hypothermic storage medium (e.g., Optisol™) is designed be to stored and used at 2-8° C., with expiration dating being impacted after 15 days at 25° C. As described in U.S. Pat. No. 5,104,787, incorporated by reference herein for its teachings regarding corneal storage media), hypothermic corneal storage media provide for enhancement of corneal tissue viability by maintaining normal physiologic metabolism and corneal deturgescence during low temperature storage, and typically contain one or more cell nutrient supplements which maintain and enhance the preservation of eye tissues, including human corneal tissues at low temperatures (2° C. to 15° C.), to maintain the attributes of fresh tissue. Optisol™, for example, contains: an aqueous nutrient and electrolyte solution; a glycosaminoglycan; a deturgescent agent; an energy source; a buffer system; an antioxidant; a membrane stabilizing component; an antibiotic and/or antimycotic; and ATP precursors, in a combination effective in maintaining corneal deturgescence, thickness, and transparency.

Using hypothermic corneal storage media (e.g., Optisol™ or Life4C™), it is recommended that the corneas be allowed to warm for at least three hours at room temperature to achieve image quality conducive to quality assessment (see, e.g., Eye Bank Corneal Endothelium Image Capture and Analysis Methods; Best Practices, by Beth Ann Benetz, Professor of Opthalmology, Case Western Reserve University, EEBA Webinar, 24 Sep. 2014).

Quality assessment factors include endothelial cell size variability (polymegathism), endothelial cell shape variability (pleomorphism), general tissue quality, presence of excessive endothelium loss (guttata; guttae), and cell count (density; cells/mm²). Obtaining an accurate endothelial cell density (ECD) is a particularly important factor for quality assessment of harvested corneal tissue. Assessment of evidence indicating endothelial disease is also performed as part of the quality assessment.

It is not always possible to obtain good quality photomicrographs even immediately on the eyes' arrival in the eye bank. Moreover, various types of problems or errors can occur during corneal tissue assessment, including poor image quality, presence of guttae, inadequate boundary tracing, forceps trauma, corneal stress lines (induced by corneal folding during excision may mimic guttae), folds from edema/temperature changes, and counting errors. Fixed-frame analysis of cell density is more prone to error than variable frame analysis. Since cell boundary tracing facilitates accurate cell counting, poor image quality makes tracing, sampling and counting more difficult and less accurate. Failure to count all cells results in low cell count.

The present disclosure surprisingly satisfies these and other needs for faster and more accurate methods for assessing corneal tissue quality for purposes of improving outcomes with respect to corneal tissue storage, cutting and transplantation.

### SUMMARY OF THE INVENTION

According to particular aspects, the temperature of human donor cornea preserved in corneal cold-storage media (e.g., at a temperature in the range of 2-8° C.) is raised (e.g., by natural or forced convection) to a temperature above ambient/room temperature (e.g., to a temperature above about 20-23° C., and up to about body temperature (e.g., about 37° C.)) for analysis of transplantability (corneal tissue quality assessment), thereby providing at least 3 distinct benefits.

First, the methods provide for a decreased timeframe for analysis of human donor corneas after cold storage. The traditional process of free convection at room temperature takes 2-3 hours on average before the donor cornea's endothelium is analyzable. Surprisingly, using Applicants' method of convection in/at an environmental temperature of up to about 37° C., that timeframe is reduced to, for example, about 0.75-1.5 hours on average, as disclosed and supported herein.

Second, the methods provide for increased endothelial cell border definition. As stated above, it is generally recognized in the art that even warming of corneas above room temperature in hypothermic storage medium has a deleterious effect on cell morphology and that folds induced by swelling of corneal tissue at such elevated temperatures results in endothelial cell damage and some cell loss. Human donor cornea endothelia are thus typically analyzed at room temperature, but where it is commonly difficult to analyze due to poorly defined cell borders. Applicants have surprising found that incubating cornea at temperatures above room temperature, and raising the temperature of the corneal tissue above room temperature, results in substantially improved tissue quality assessment. Without being bound by mechanism, at temperatures above room temperature and up to about natural body temperature (e.g., about 37° C.) the donor cornea endothelia appear to respond more typical of in vivo cells, creating better resolution of the cell borders. Moreover, with better defined borders and better specular reflection, a larger quantity of cells are elucidated, resulting in a higher and more accurate cell density analysis, as disclosed and supported herein in the working examples. Applicants, therefore, despite the conventional wisdom that incubation of corneal tissue after hypothermic storage should not exceed room temperature (to avoid tissue damage), have found that incubating corneal tissue in hypothermic corneal storage media to and/or at a temperature above ambient temperature (e.g., to and/or at a temperature approximating body temperature or approximating the cornea's natural functional temperature), results in substantially improved tissue quality assessment, while maintaining tissue quality and viability for transplantation. The discovery of the capacity of the tissue to maintain such quality and viability at tissue analysis above ambient temperature, and even after one or more periods of cold storage (e.g., at a temperature in the range of 2-8° C.), provides for new and useful methods over the prior art, as claimed herein.

Third, the methods provide for an increased transplant rate. According to particular aspects, a percentage of human donor cornea endothelia are not able to achieve satisfactory specular reflection or definition of cell borders at room temperature. Such corneas are then typically determined not suitable for transplant (NSFT), either for low cell density or poor endo/dropout. According to additional aspects, at temperatures above room temperature (e.g., up to about natural body temperature) some of these cells are better elucidated by achieving a metabolically active status, resulting in specular reflection congruent with appropriate (better) analysis, as disclosed and supported herein in the working examples.

The results are surprising and unexpected given the art-recognized contra-indication for warming of corneas above room temperature in hypothermic storage medium, because of deleterious effects on cell morphology, and folds induced by swelling of corneal tissue at such elevated temperatures resulting in endothelial cell damage and some cell loss. Surprisingly, active warming (e.g., convection incubator) at temperatures above room temperature, to bring the corneal tissue to a temperature above room temperature, provides the surprising advantages disclosed and supported herein.

### BRIEF DESCRIPTION OF THE DRAWINGS

FIGS. 1A and 1B show, according to particular aspects, poor photo quality when human corneal tissue is analyzed at room temperature to determine the Endothelial Cell Density (ECD), and where the ECD was estimated to be 2190 cells/mm².

FIGS. 2A and 2B show, according to particular aspects, that incubating the same human corneal tissue as shown in the room temperature ECD analysis of FIGS. 1A and 1B at 34° C. (about body temperature) for 0.75-1.5 hours on average, resulted in substantially improved photo quality, where the Endothelial Cell Density (ECD) was estimated to be 2729 cells/mm².

FIGS. 3A and 3B show, according to particular aspects, poor photo quality (slit lamp) when human corneal tissue is analyzed at room temperature (3A), versus with incubation at 34° C. (3B), to determine the Dropout (endothelial cell loss). Patches of cells in 3A deemed non-viable at room temperature are nonetheless deemed viable when the human corneal tissue is analyzed with incubation at 34° C. for 0.75-1.5 hours on average.

FIGS. 4A and 4B show, according to particular aspects, poor photo quality when human corneal tissue is analyzed at room temperature (4A), versus with incubation at 34° C. (4B), to determine the endothelial quality (proper reflection of light by endothelial cells). Many cells in 4A deemed non-viable at room temperature because they fail to flatten to reflect light, are nonetheless deemed viable when the human corneal tissue is analyzed with incubation at 34° C. for 0.75-1.5 hours on average, where, according to particular aspects, the corneal endothelial cells are sufficiently metabolically active to lie flat.

FIG. 5 shows, according to particular aspects, that in addition to the benefits derived from holding the cornea at temperatures above room temperature (e.g., incubating the corneal tissue at about body temperature) for evaluation, placing the corneal tissue in an environmental temperature above room temperature when initially removing the corneal tissue from the tissue fridge at 2-8° C., results in the cornea more quickly warming to a temperature conducive with optimal endothelial evaluation, thus providing a significant time saver. Exemplary Environmental Temperatures are as follows: top curve, 50° C.; middle curve 34° C.; and lower curve 23° C.

### DETAILED DESCRIPTION OF THE INVENTION

Particular aspects provide methods for assessing corneal tissue quality, comprising: incubating corneal tissue in hypothermic corneal storage media to a temperature above ambient temperature; and assessing, using suitable microscopic examination, endothelial cell density and/or morphology and/or loss.

The methods may additionally comprise storing the corneal tissue in the hypothermic corneal storage media at a temperature in the range of 2-8° C. prior to, and/or after assessing the corneal tissue quality by incubating the corneal tissue at the temperature above ambient temperature.

The methods may additionally comprise: incubating the corneal tissue in the hypothermic corneal storage media at ambient temperature, and initially assessing, using suitable microscopic examination, endothelial cell density and/or morphology and/or loss at ambient temperature; and storing the initially assessed corneal tissue in the hypothermic corneal storage media at a temperature in the range of 2-8° C. prior to assessing the corneal tissue quality by incubating the corneal tissue at the temperature above ambient temperature.

The methods may additionally comprise: after assessing corneal tissue quality at the temperature above ambient temperature, storing the assessed corneal tissue in the hypothermic corneal storage media at a temperature in the range of 2-8° C., and then re-assessing the corneal tissue quality by incubating the corneal tissue at the temperature above ambient temperature.

In the methods, incubating the corneal tissue in the hypothermic corneal storage media to the temperature above ambient temperature is for a time period sufficient to enhance endothelial cell border definition relative to that seen at ambient temperature.

In the methods, the time period sufficient to enhance endothelial cell border definition relative to that seen at ambient temperature is, in particular aspects, a time period selected from a time-range group consisting of 0.5-6 hr., 0.5 to 5 hr., 0.5 to 4 hr., 0.5 to 3 hr., 0.5 to 2 hr., 0.5 to 1.5 hr., 0.5 to 1 hr., and 0.7 to 1.5 hr.

In particular aspects of the methods, microscopic assessment comprises, for example, specular microscopy and/or slit-lamp microscopy.

In particular aspects of the methods, ambient temperature is a temperature in a range selected from the group consisting of from 16° C. (approximately 61° F.) to about 24° C. (approximately 75° F.), from about 20° C. (68° F.) to about 23° C. (approximately 73° F.), from about 20° C. to 22° C. (68° F.-72° F.), and from about 15° C. (59° F.) to about 22° C. (72° F.).

In particular aspects of the methods, incubating corneal tissue to a temperature above ambient temperature comprises incubating the corneal tissue at a temperature approximating body temperature or approximating the cornea's natural functional temperature (e.g., comprises incubating the corneal tissue at a temperature in a range selected from the group consisting of from 25° C. (approximately 75° F.) to 38° C. (approximately 100° F.), 30° C. (approximately 86° F.) to 38° C. (approximately 100° F.), from 31° C. (approximately 89° F.) to 37° C. (approximately 99° F.), from 32° C. (approximately 90° F.) to 36° C. (approximately 97° F.), from about 33° C. (approximately 91° F.) to 36° C. (approximately 97° F.), from 33° C. (approximately 91° F.) to 35° C. (approximately 95° F.), and from 33° C. (approximately 91° F.) to 34° C. (approximately 93° F.), for at least 0.5 hours).

In particular aspects of the methods, incubating corneal tissue to a temperature above ambient temperature or approximating the cornea's natural functional temperature comprises incubating the corneal tissue to a temperature sufficient to enhance endothelial cell border definition relative to that seen at ambient temperature.

In particular aspects of the methods, incubating corneal tissue to a temperature above ambient temperature or approximating the cornea's natural functional temperature comprises incubating the corneal tissue to a temperature in the range of 27 (approximately 81° F.) to 38 (approximately 100° F.), 27 (approximately 81° F.) to 37 (approximately 99° F.), 27 (approximately 81° F.) to 36 (approximately 97° F.), 27 (approximately 81° F.) to 35 (approximately 95° F.), and 27 (approximately 81° F.) to 34 (approximately 93° F.).

The methods may further comprise validating the corneal tissue for transplantation purposes, based on the assessed corneal tissue quality.

In particular aspects of the methods, the hypothermic corneal storage media comprises at least one ingredient selected from the group consisting of: an aqueous nutrient and electrolyte solution; a glycosaminoglycan; a deturgescent agent; an energy source; a buffer system; an antioxidant; a membrane stabilizing component; an antibiotic and/or antimycotic; and ATP precursors, in a combination effective in maintaining corneal deturgescence, thickness, and transparency.

In particular aspects of the methods, the hypothermic corneal storage media comprises at least one selected from the group consisting of: Optisol™; Optisol™ GS; Dexsol™; Life4C™; McCarey-Kaufman™ (M.K.); and Eusol-C™.

In particular aspects of the methods, wherein the hypothermic corneal storage media comprises chondroitin sulfate and/or dextran 40.

The methods are generally applicable to humans and animals having corneas, preferably to mammalian corneal tissue (e.g., humans, dogs, etc.).

Additional aspects provide a validated corneal tissue sample, validated for transplantation purposes using a method for assessing corneal tissue quality according to any of the methods disclosed herein. Preferably, the corneal tissue is human.

### Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew, et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratis, and is used herein to indicate a non-limiting example. Thus, the abbreviation “e.g.” is synonymous with the term “for example. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention such as the biological activity of the claimed composition or method. Embodiments defined by each of these transition terms are within the scope of this invention.

In order to facilitate review of the various embodiments of this disclosure, the following explanations of specific terms are provided:

The term “about” when used before a numerical value indicates that the value may vary within a reasonable range, such as +5%, +4%, +3%, +2%, +1%, and +0.2%.

In the context of this disclosure “room temperature” refers to any temperature within a range of temperatures between about 16° C. (approximately 61° F.) and about 25° C. (approximately 77° F.). Commonly, room temperature is between about 20° C. and 22° C. (68° F.-72° F.). Generally, the term room temperature is used to indicate that no additional energy is expended cooling (e.g., refrigerating) or heating the sample or ambient temperature.

“Ambient temperature” or “room temperature” is a temperature in a range selected from the group consisting of from 16° C. (approximately 61° F.) to about 24° C. (approximately 75° F.), from about 20° C. (68° F.) to about 23° C. (approximately 73° F.), from about 20° C. to 22° C. (68° F.-72° F.), and 15° C. (59° F.) and 22° C. (72° F.).

“Physiological temperature” or “body temperature” or “temperature or approximating the cornea's natural functional temperature” is a temperature in the range of 27° C. (approximately 81° F.) to 38° C. (approximately 100° F.), 27° C. (approximately 81° F.) to 37° C. (approximately 99° F.), 27° C. (approximately 81° F.) to 36° C. (approximately 97° F.), 27° C. (approximately 81° F.) to 35° C. (approximately 95° F.), and 27° C. (approximately 81° F.) to 34° C. (approximately 93° F.). In the working examples herein, physiological or body temperature is typically 34° C.

“Deturgescence”, as recognized in the art, is essential to prevent excessive hydration of cornea from an influx of cations and water molecules into the corneal stroma (corneal swelling/edema), which in turn could result in progressive corneal opacity or cloudiness, leading to functional blindness. Deturgescence is therefore essential to maintain a clear, transparent cornea by relative corneal dehydration through the action of the impermeable epithelium as well as through the metabolic transport system in the endothelium. Exemplary deturgescent agents include, but are not limited to dextran (e.g., dextran 40, dextran 70, and/or dextran 500), dextran sulfate, chondroitin sulfate, NaCl, dextrose, sucrose, other sugars, and combinations of the preceding examples.

Applicant's “minimum standards for transplantation” as used herein refers to a minimum of 2000 corneal endothelial cells per mm², measured by specular microscope; and where cell “dropout”, if present, is classified as mild, mild to moderate, moderate, or severe, and where the cell dropout rating may be used in combination with the cell density to validate or preclude the cornea from being used for transplant regardless of the cell density.

“NSFT: as used herein refers to not suitable for transplantation, according to Applicant's minimum standards for transplantation.

The adjective “pharmaceutically acceptable” indicates that the subject is physiologically acceptable for administration to a subject (e.g., a human or animal subject). Remington's Pharmaceutical Sciences, by E. W. Martin, Mack Publishing Co., Easton, Pa., 15th Edition (1975), describes compositions and formulations (including diluents) suitable for pharmaceutical delivery of therapeutic and/or prophylactic compositions, including vaccines.

The phrase “Good Manufacturing Practice” or “GMP” with respect to methods and procedures employed in vaccine production refer specifically to the set of methods, protocols and procedures established by the United States Food and Drug Administration (FDA). Similar recommendations and guidelines are promulgated by the World Health Organization. The abbreviation “cGMP” specifically designates those protocols and procedures that are currently approved by the FDA (e.g., under 21 Code of Federal Regulations, parts 210 and 211, available on the world wide web at fda.gov/cder/dmpq). With time cGMP compliant procedures may change. Any methods disclosed herein can be adapted in accordance with new cGMP requirements as mandated by the FDA.

It will be apparent that the precise details of the methods or compositions described can be varied or modified without departing from the spirit of the described invention. The following examples are provided to illustrate certain particular features and/or embodiments. These examples should not be construed to limit the invention to the particular features or embodiments described. Each of the references cited below is incorporated by reference for all purposes.

### Example 1

### Exemplary Materials

Corneal Tissue.

Human donor corneal-scleral rims were used.

Hypothermic Storage Media.

Optisol™ GS was used for the present working examples experiments. Other exemplary media that Applicant's incubation technique encompasses are, e.g., Life4C.™, McCarey-Kaufman™ Media, EUSOL-C™, Dexsol™, K-Sol®, CSM™, Chen Medium™, Cornisol™, Steinhardt Media™, Likorol™, etc.

Corneal Preservation Chamber.

Exemplary corneal preservation chambers include, but are not limited to, for example, Krolman Viewing Chamber (as used in the present working examples), Bausch&Lomb Viewing Chamber, Numedis Transend Chamber, Alcon Viewing Chamber. Preservation in the original media chamber is also an option.

Specular Microscope.

HAI CAS EB-3000xyz (used for our experiments), Konan EB-10, or other suitable specular microscope.

Incubator.

Thermo-Scientific Heratherm Compact Incubator, or other suitable incubator

Slit-Lamp Microscope.

A Topcon SL-D7 slit-lamb microscope was used in the present working examples. Other slit-lamp microscopes are also encompassed within the scope of the present invention.

### Example 2

### Exemplary Re-Evaluation, and Initial Evaluation Methods were Successfully Applied

EyeBank Specular Microscopy.

An overview of EyeBank Specular Microscopy is available on-line at “slideshare.net/EBAICME/eye-bank-specular-microscopy”.

Both re-evaluation and initial evaluation were performed as follows:

A—Re-Evaluation by Incubation at 34° C. in Optisol™ GS after an Initial Evaluation at Room Temperature:

According to particular aspects of the present invention, after an initial evaluation at room temperature, the endothelial cell response of the initially evaluated corneas was re-evaluated by incubating poor endo, moderate dropout & poor photo quality corneas at 34° C. in Optisol™ GS as follows:
a. An initial evaluation using specular microscopy and slit-lamp microscopy is performed at room temperature as soon as possible after recovery of donor corneal-scleral rims. If it is determined that the donor cornea endothelium does not meet the minimum requirements for transplantation due to poor image quality, poor endothelium or dropout, then the cornea is stored in the tissue fridge overnight for re-evaluation the next day.
b. The endothelial re-evaluation (1-day after initial evaluation) is conducted with the donor cornea incubated at a temperature of 34° C.
c. Corneas for re-evaluation are removed from the tissue fridge and placed in, for example, a Thermo-Scientific Heratherm Incubator set at a temperature of 34° C. Checking every 0.5-1 hrs, the Specular and Slit-lamp Microscope Technician removes and re-evaluates the tissue once the endothelium has achieved a satisfactory quality with well-defined cell borders (typically about 0.7 to about 1.5 hours, or longer if necessary).
d. If the corneal endothelium has achieved Applicant's minimum standards for transplantation (e.g., a minimum of 2000 corneal endothelial cells per mm², measured by specular microscope; and where cell “dropout”, if present, is classified as mild, mild to moderate, moderate, or severe, and where the cell dropout rating may be used in combination with the cell density to validate or preclude the cornea from being used for transplant regardless of the cell density).
e. If the tissue has not achieved a level of endothelial cell quality conducive with an accurate evaluation by the end of day or 6 hours at 34° C., the tissue should be deemed/rejected as not suitable for transplant (NSFT) for poor endo (poor endothelial cell density). If the cell quality is conducive with an accurate evaluation but the endothelium still exhibits excessive cell loss or guttae, it should be deemed/rejected as ? (as questionable)) NSFT for dropout.

According to particular aspects of the present invention, and as shown in the working examples below, a portion of poor image quality, poor endo or dropout grade donor cornea endothelia, as initially judged by room temperature evaluation, achieved a state of better cell reflection and better cell border definition when evaluated at the cornea's natural functional temperature (e.g., about 34° C.). While some corneas appeared to have endothelial dysfunction at room temperature, a portion of these exhibited evidence of cell viability equivalent to or better than Applicant's minimum standards for transplantation (see above).

B—Initial-Evaluation by Incubation at 34° C. in Optisol™ GS:

According to additional aspects of the present invention, the endothelial cell response of corneas, that were going to be processed for e.g., Descemet's Membrane Automated Endothelial Keratoplasty (DSAEK), Descemet's Membrane Endothelial Keratosplaty (DMEK) or Intralase Enabled Keratoplasty (IEK), was initially evaluated (no prior room temperature assessment) by incubating corneas at 34° C. in Optisol™ GS until their endothelial cells are optimally analyzable (typically about 0.7 to about 1.5 hours at 34° C.) as follows:

a—Corneas suitable for processing are removed from the tissue fridge and placed in a Thermo-Scientific Heratherm Incubator set at a temperature of 34° C. Checking every 0.5-1 hrs, the Processing Technician can remove and evaluate the tissue once the endothelium has achieved a satisfactory quality for processing (typically about 0.7 to about 1.5 hours, or longer if necessary);

b—If the corneal endothelium has achieved Applicants' minimum standards for transplantation (see above), processing for DSAEK/DMEK/IEK should be continued;

c—If the tissue has not achieved a level of endothelial cell quality conducive with an accurate evaluation by the end of day, the tissue should be deemed/rejected as NSFT for poor endo (endothelial cell density). If the cell quality is conducive with an accurate evaluation but the endothelium exhibits excessive cell loss or guttata, it should be deemed/rejected as NSFT for dropout;

d—Any adverse reactions from incubated and processed tissue is tracked.

According to particular aspects, and based on working data and examples herein, the average percentage of approved corneas deemed not suitable for processing prior to cutting/processing significantly decreased, including in comparison of historical averages to current averages using incubation at temperatures above room temperature (e.g., 34° C.).

### Example 3

### Re-Evaluation of Corneas at 34° C. Resulted in Identifying Corneas as Transplantable, Whereas the Same Corneas were Deemed not Suitable for Transplant (NSFT) Because of Poor Image Quality (Poor Endothelial Density) Upon Initial Evaluation of the Same Cornea at Room Temperature as in the Prior Art

As described above under “Background”, obtaining an accurate endothelial cell density (ECD) is a particularly important factor for quality assessment of harvested corneal tissue.

This example shows that at least a portion of poor image quality, poor endo (or dropout grade as shown in working Example 4 below) donor cornea endothelia, as initially judged at room temperature, achieved a state of better cell reflection and better cell border definition when evaluated at the cornea's natural functional temperature (e.g., 34° C.). While some corneas appeared to have endothelial dysfunction at room temperature, a portion of these exhibited evidence of cell viability equivalent to or better than Applicant's minimum standards for transplantation (e.g., a minimum of 2000 corneal endothelial cells per mm², measured by specular microscope; and where cell “dropout”, if present, is classified as mild, mild to moderate, moderate, or severe, and where the cell dropout rating may be used in combination with the cell density to validate or preclude the cornea from being used for transplant regardless of the cell density).

While the definitions of Poor Endo/Dropout/Bad Photo Quality can be somewhat conflated, these are based on Photo Quality Guidelines reference: PDF “Benetz_EBAA Webinar 24 Sep. 2014, and were determined by Applicants to be the best categories for division of re-evaluation data collection (e.g., at 34° C. in Optisol™ GS).”

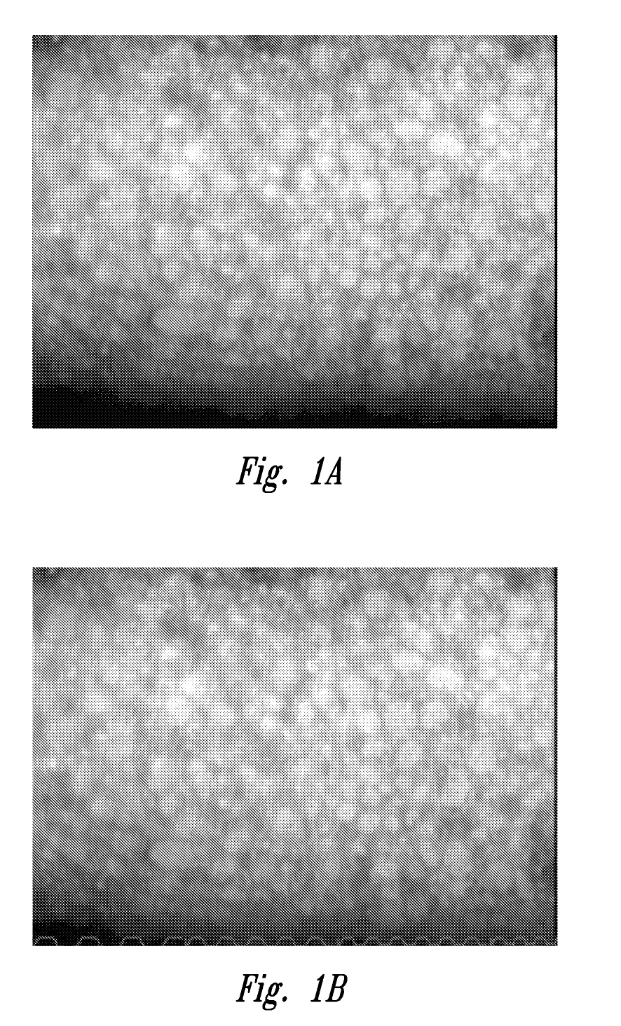

An example of a poor photo quality cornea analyzed at room temperature is shown in FIGS. 1A and 1B, where endothelial cell density has been measured as 2190 cells/mm².

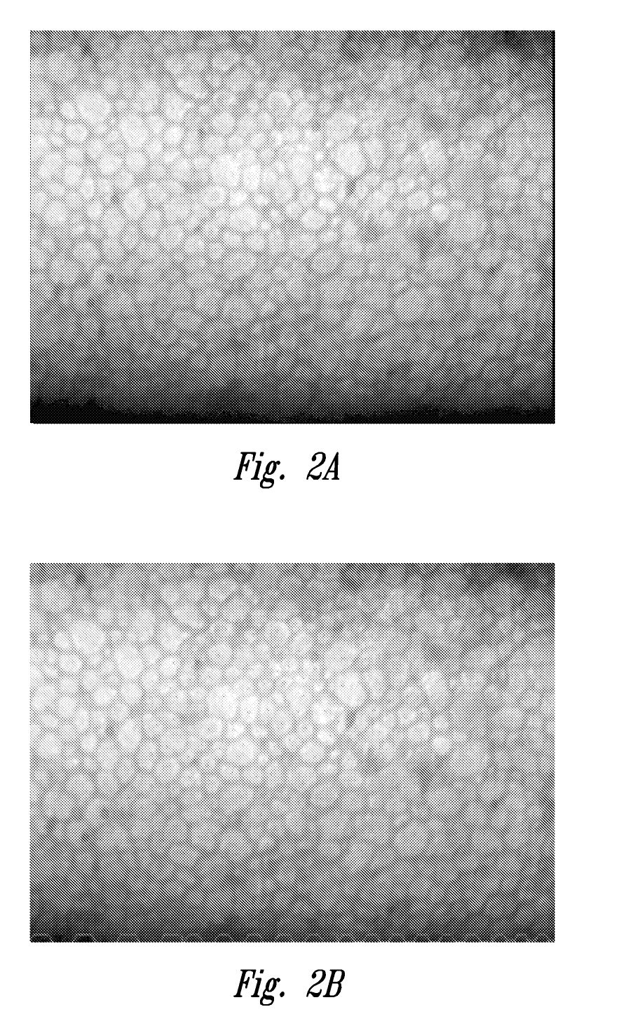

An example of a good photo quality cornea incubated/analyzed at the cornea's natural functional temperature (e.g., 34° C.) is shown in FIGS. 2A and 2B, where endothelial cell density has been measured as 2729 cells/mm². This is the same cornea used in the analysis of FIGS. 1A and 1B that was deemed as not transplantable (NSFT), but here initially examined with 34° C. incubation.

As can be seen from FIGS. 1A and 1B, there are cells that somewhat exhibit evidence of viability, but many lack well-defined cell borders. In the past, while this particular cornea might have been approved for transplant, it more likely would not have been approved because of an under-reported cell density due to inability to distinguish individual cells. By contrast, in FIGS. 2A and 2B, with incubation at the cornea's natural functional temperature (e.g., 34° C.), the cell borders are much better defined, which allowed a more accurate analysis cell density without much “guess work.”

Of 250 human corneal samples examined, only 25 were deemed not NSFT after re-evaluation by Applicant's re-evaluation methods using incubation at the cornea's natural functional temperature (e.g., 34° C.), thus representing a 90% success rate.

### Example 4

### Re-Evaluation of Corneas at 34° C. Resulted in Identifying Corneas as Transplantable, Whereas the Same Corneas were Deemed not Suitable for Transplant (NSFT) Because of Dropout Status Upon Initial Evaluation of the Same Cornea at Room Temperature as in the Prior Art

Dropout.

Dropout is a term used during a qualitative analysis of the endothelium using a slit-lamp, and represents cells or patches of cells that do not reflect light, and are thus regarded as non-viable cells.

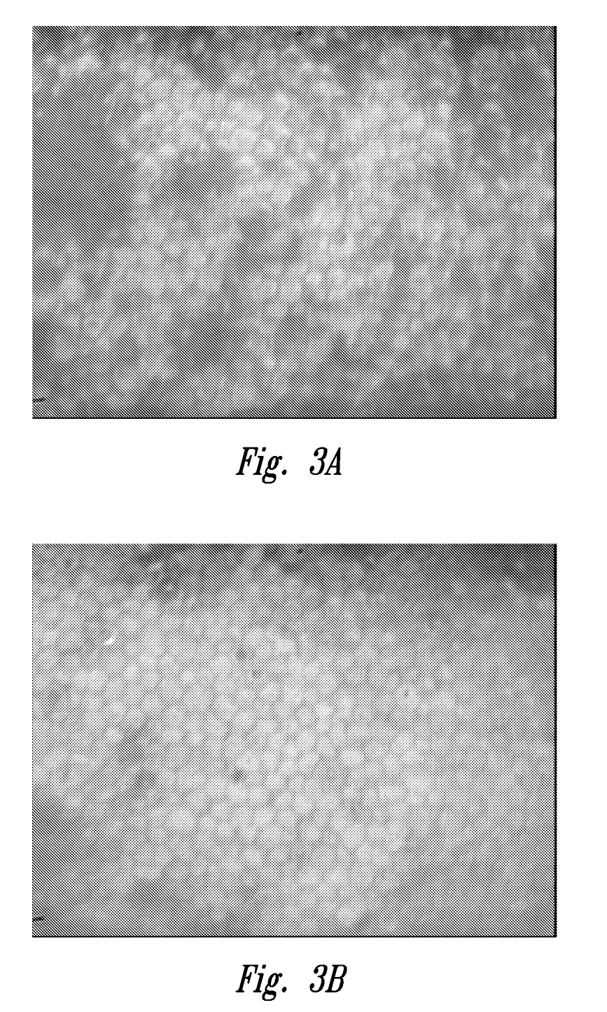

An example of dropout quality is shown in FIGS. 3A (room temperature analysis) and 3B (34° C. incubation), showing what appear to be cells or patches of cells are not reflecting light when analyzed using prior art room temperature methods, and would be construed as excessive loss of endothelial cells; that is, the endothelial cells are not viable, have lysed, or there are guttae. Guttae are sub-endothelial deposits that create a little bump in the Descemet's Membrane (DM). The endothelial cells still reside on that section of the DM, but aren't visible when the rest of the endothelium is in focus during specular microscopy due to the thickness aberration. If there are enough guttae then the graft is deemed unusable for endothelial transplant.

According to particular aspects illustrated in this example, however, when a slit-lamp technician sees such an apparent excessive loss of endothelial cells, such a cornea would be re-evaluated at 34° C.

Of 190 human corneal samples examined, only 63 were deemed not NSFT after re-evaluation by Applicant's re-evaluation methods, thus representing a 67% success rate.

### Example 5

### Re-Evaluation of Corneas at 34° C. Resulted in Identifying Corneas as Transplantable, Whereas the Same Corneas were Deemed not Suitable for Transplant (NSFT) Because of Poor Endothelium Status Upon Initial Evaluation of the Same Cornea at Room Temperature as in the Prior Art

Poor Endothelium.

Poor endothelium corneas have endothelial cells that don't flatten to reflect light. In many cases poor endothelium and dropout grades may be used somewhat interchangeably.

Poor endothelium can also be regarded as the most severe grade of dropout when there are almost no cells reflecting light appropriately. When a corneal endothelial cell is metabolically active it lays flat against the Descemet's Membrane (DM) and is pumping water out of the stroma.

Applicant noted that these poor endothelium corneas have visible cells that are only apparently not viable, as judged by prior art room temperature methods. Applicant determined, surprisingly, that with Trypan blue staining there is little or no cell loss, and hypothesized that some donor cornea endothelia have a higher temperature threshold for sustained metabolic activity, and if that threshold temperature isn't attained, the cells regress to a state of energy conservation, similar to that observed at 2-8° C.

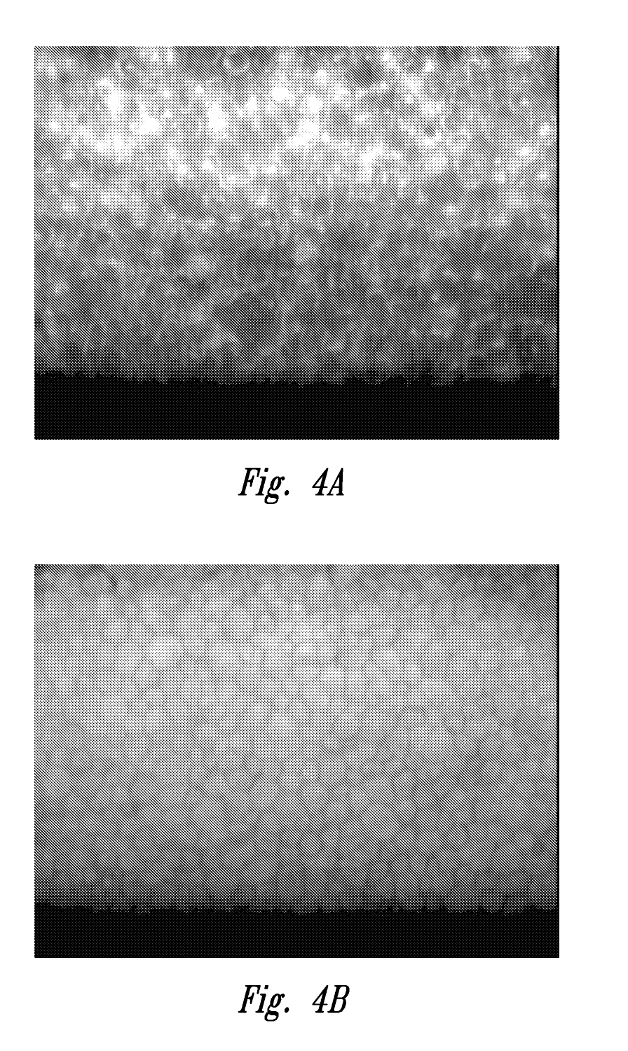

FIG. 4A shows a poor endothelium cornea analyzed by prior art room temperature methods.

FIG. 4B shows the same cornea analyzed with incubation at the cornea's natural functional temperature (e.g., 34° C.) by Applicant's method, and where the viable endothelial call are readily visible.

Of 133 human corneal samples examined, only 47 were deemed not NSFT after re-evaluation by Applicant's re-evaluation methods, thus representing a 65% success rate.

### Example 6

### Pre-Processing Corneas were Initially Incubated at 34° C. (No Prior Room Temperature Analysis) Until their Endothelial Cells are Analyzable, Resulting in a Marked Improvement Over Prior Art Room Temperature Methods

According to particular aspects of the present invention, along with the re-evaluations of tissue with perceived endothelial dysfunction based on prior art room temperature methods (as outlined under Methods in working examples 2-5 above), all pre-processing corneas are initially incubated at 34° C. (without any prior room temperature analysis) until their endothelial cells are analyzable.

As outlined under Example 2, method B, and according to additional aspects of the present invention, the endothelial cell response of corneas, that were going to be processed for e.g., Descemet's Membrane Automated Endothelial Keratoplasty (DSAEK), Descemet's Membrane Endothelial Keratosplaty (DMEK) or Intralase Enabled Keratoplasty (IEK), was initially evaluated (no prior room temperature assessment) by incubating corneas at 34° C. in Optisol™ GS until their endothelial cells are optimally analyzable (typically about 0.7 to about 1.5 hours at 34° C.) Corneas suitable for processing were removed from the tissue fridge and placed in the Thermo-Scientific Heratherm Incubator set at a temperature of 34° C. Checking every 0.5-1 hrs, the Processing Technician removed and evaluated the tissue once the endothelium achieved a satisfactory quality for processing (e.g., DSAEK/DMEK/IEK).

According to particular aspects of the present invention, and based on the working examples, the average percentage of approved corneas deemed not suitable for processing prior was significantly reduced. Specifically, out of approximately 700 corneas incubated prior to processing, the prior method of letting corneas reach room temperature for the pre-processing evaluation yielded 8% of endothelial evaluations not being approved. By contrast, with the new procedure of incubation at 34° C. prior to processing, this percentage was around 4%.

While not being bound by mechanism, Applicant postulates that with evaluation at room temperature the endothelial cells sometimes aren't reaching a minimum metabolic threshold and are remaining in an energy conserving hypothermic-induced dormant state. Incubation at body temperature likely promotes achieving a minimum metabolic threshold required for accurate endothelial cell analysis as shown in the working examples herein. Donor cornea endothelia achieved a state of better cell reflection and better cell border definition when evaluated at the cornea's natural functional temperature (e.g., 34° C.). A significant portion of these donor corneas would appear to have endothelial dysfunction, if analyzed by prior art room temperature methods.

### Example 7

### Pre-Processing Corneas were Initially Examined by Room Temperature Analysis, and then Re-Examined, if Necessary, by Either Room-Temperature Evaluation, or Evaluation Using Incubation at the Cornea's Natural Functional Temperature (e.g., 34° C.), and where Above Room Temperature Incubation Resulted in a Marked Improvement Over Prior Art Room Temperature Methods

For the data of this working example, corneal tissues were incubated in two situations:
When a corneal tissue needed to be re-evaluated for endothelial reasons (Normal evaluation process); or
When the corneal tissue was removed from the fridge to be warmed for pre-cutting (incubator evaluation process).

### Normal Evaluation Process:

1. Remove tissue from fridge and place at room temperature. Start evaluation approximately 1.5-3 hours after removal from fridge. Some corneas may take all day to warm up properly. There are 4 possible outcomes by the end of that day:
a—The cornea has a suitable slit-lamp and specular photo, and is approved for transplant;
b—The cornea is not suitable for transplant, and the reason is deemed to be relatively definitive (e.g., presence of an infiltrate, tech induced damage, or a low cell count where there is a clear photo), and where a second person confirms the pathology, and the cornea is deemed NSFT;
c—There is a non-endothelial issue that needs another opinion by a more experienced evaluator, or a consult, and approval/NSFT is held off until that opinion is procured (e.g., either in person or by photo consult);
d—If, at the end of the day, we are not satisfied with our evaluation from an endothelial perspective, such as not being able to get a good quality photo, in the case of poor endo, or think that we are not getting good visualization of the cell dropout, we would place the cornea back in the fridge to be re-evaluated again a day later.

2. If a cornea falls into 1.d.: All of the corneas that fall into part d. will get evaluated the next day at room temperature, and a final determination will be made.

1-7 days later, a cornea that is cleared for transplant is selected to be cut. The cornea is pulled from the fridge and placed at room temperature to warm prior to cut. On average, corneas will warm for at least 2 hours prior to cutting.

Incubator Re-Evaluation Process, for this Study:

1. Same as above.

2. If a cornea falls into 1.d.: The cornea is taken from the fridge and placed directly in the incubator at 34° C. for re-evaluation. Timers are set for every 30 minutes to ensure that corneas are checked regularly, and not incubated for longer than needed. Corneas, after pulling from the fridge can be ready as early as 0.5 to 0.7 hours after placing them in the incubator.

1-7 days later, a cornea that is cleared for transplant is selected to be cut. The cornea is pulled from the fridge and placed in the incubator at 34° C. to warm prior to cut. Timers are set for every 30 minutes to ensure that corneas are checked regularly, and not incubated for longer than needed. Corneas, after pulling from the fridge can be ready as early as 0.5 to 0.7 hours after placing them in the incubator 34° C. for re-evaluation.

Both control (historical room temperature evaluation/re-evaluation), and incubator corneal tissue re-evaluation data were compared. The historical control group was selected to provide a similar sized sample that had the closest possible donor population to the incubator group, and the same tissue sources were used throughout the time periods corresponding to the respective control and incubator re-evaluation data gathering.

### Re-Evaluation (Incubation at 34° C.) Cornea Results:

Of 789 corneas that were re-evaluated at 34° C., 748 of them completed the re-evaluation process, and 35 dropped out of the study because they became NSFT for another reason, usually medical review related. Information about the 748 that completed the study is as follows:

| Reason for re-eval | Number | % of total | NSFT | Eval discard rate |
|---|---|---|---|---|
| bad photo quality | 343 | 45.86% | 56 | 16.33% |
| dropout | 241 | 32.22% | 95 | 39.42% |
| poor endo | 155 | 20.72% | 59 | 38.06% |
| spec not done | 9 | 1.20% | 5 | 55.56% |
| Total | 748 | | 215 | 28.74% |

Therefore, about 71% of the corneas re-evaluated in the incubator were successful, and approved for penetrating keratoplasty (PK) or endothelial keratoplasty (EK), which was a much higher success rate than Applicants' historical re-evaluation practice, without the incubator. The incubator data was compared to the control data in two ways.

First, Applicants' traditional evaluation discard metric was analyzed as a percent of all corneas recovered with the intent for transplant, and looking at the non-tech endo reasons only, the following statistics were observed:

| Endo related | | Total | | | |
|---|---|---|---|---|---|
| eval discards | | corneas | % NSFT | Lower limit CI | Upper limit CI |
| incubator | 330 | 4581 | 7.20% | 6.45% | 7.95% |
| control | 777 | 6147 | 12.64% | 11.81% | 13.47% |

Statistically significant, p<0.05. No overlap in the 95% confidence intervals.

Based on this, the evaluation discard rate was reduced by approximately 5%, and the difference is statistically significant. This does include all corneas evaluated during the time period that the incubator was in use, even though the majority of the corneas were not incubated.

Second, to get more directly at the re-evaluation process, we looked only at tissues that had been re-evaluated and compares the success rates there, using data points in both the incubator and control group, with the following re-evaluation criteria:
The suitability was completed 1 day or more after the slit-lamp exam; and
The specular evaluation was not completed on the same day as the slit-lamp exam.
   While not a perfect measure, it is the closest with the available data. By using this measure in the incubator time period (for data gathering), 746 corneas were identified as re-evaluations, and 699 of those were actually incubated, providing the following results:

| Endo NSFT | | Total | % | | |
|---|---|---|---|---|---|
| Re-evals | | re-evals | NSFT | Lower limit CI | Upper limit CI |
| incubator | 176 | 746 | 23.59% | 20.55% | 26.64% |
| control | 422 | 953 | 44.28% | 41.13% | 47.43% |

Statistically significant, p<0.05. No overlap in the 95% confidence intervals.
This comparison more optimally shows the impact of incubation—reducing the amount of re-evaluation discard rate corneas by almost 50%.

### Example 8

### Decreased Warming Times Afforded by Incubating at Body Temperature Provide a Time-Saving Improvement

In addition to the benefits derived from incubating the cornea at about body temperature (e.g., at the cornea's natural functional temperature (e.g., 34° C.)) for evaluation, using applicants incubation methods, placing the corneal tissue in an environmental temperature above room temperature when initially removing the corneal tissue from the tissue fridge at 2-8° C., results in the cornea more quickly (for example, in about 0.75-1.5 hours on average, as disclosed and supported herein) warming to a temperature conducive with endothelial evaluation upon being initially removed from the tissue fridge at 2-8° C.

This time-saving improvement applies to both Applicant's initial re-evaluations and also Applicant's pre-processing evaluations.

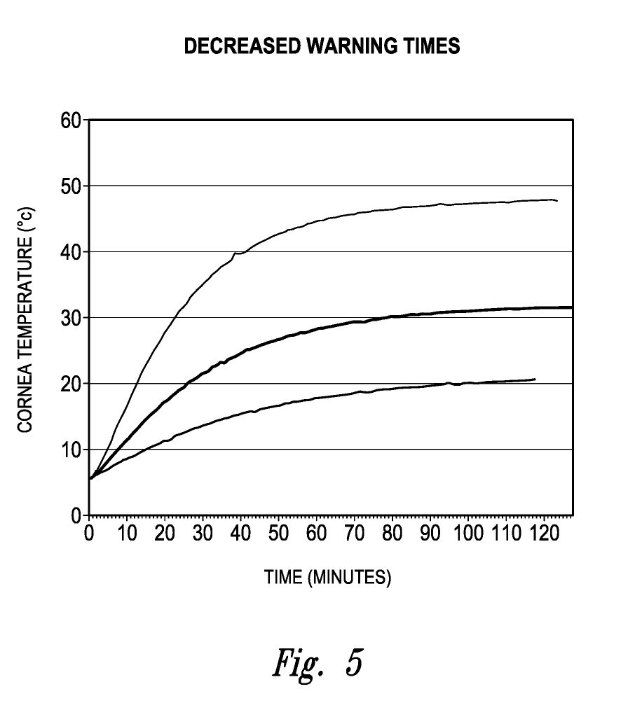

FIG. 5 shows that corneas removed from a tissue refrigerator at 2-8° C., warm to a temperature conducive with endothelial evaluation more quickly when incubated at 34° C. compared to incubation at room temperature (e.g., 23° C.). Exemplary Environmental Temperatures (for warming the corneal tissue to a temperature conducive with endothelial evaluation upon being initially removed from the tissue fridge at 2-8° C.) are as follows: top curve, 50° C.; middle curve 34° C.; and lower curve 23° C.

1. A method for assessing corneal tissue quality, comprising: incubating corneal tissue in hypothermic corneal storage media to a temperature above ambient temperature; and assessing, using suitable microscopic examination, endothelial cell density and/or morphology and/or loss.

2. The method of claim 1, additionally comprising storing the corneal tissue in the hypothermic corneal storage media at a temperature in the range of 2-8° C. prior to, and/or after assessing the corneal tissue quality by incubating the corneal tissue at the temperature above ambient temperature.

3. The method of claim 1, additionally comprising; incubating the corneal tissue in the hypothermic corneal storage media at ambient temperature, and initially assessing, using suitable microscopic examination, endothelial cell density and/or morphology and/or loss at ambient temperature, and storing the initially assessed corneal tissue in the hypothermic corneal storage media at a temperature in the range of 2-8° C. prior to assessing the corneal tissue quality by incubating the corneal tissue at the temperature above ambient temperature.

4. The method of claim 1, additionally comprising, after assessing corneal tissue quality at the temperature above ambient temperature, storing the assessed corneal tissue in the hypothermic corneal storage media at a temperature in the range of 2-8° C., and then re-assessing the corneal tissue quality by incubating the corneal tissue at the temperature above ambient temperature.

5. The method of claim 1, wherein incubating the corneal tissue in the hypothermic corneal storage media to the temperature above ambient temperature is for a time period sufficient to enhance endothelial cell border definition relative to that seen at ambient temperature.

6. The method of claim 4, wherein the time period sufficient to enhance endothelial cell border definition relative to that seen at ambient temperature is a time period selected from a time-range group consisting of 0.5 to 6 hrs., 0.5 to 5 hrs., 0.5 to 4 hrs., 0.5 to 3 hrs., 0.5 to 2 hrs., 0.5 to 1.5 hrs., 0.5 to 1 hr., and 0.7 to 1.5 hrs.

7. The method of claim 1, wherein microscopic assessment comprises specular microscopy and/or slit-lamp microscopy.

8. The method of claim 1, wherein ambient temperature is a temperature in a range selected from the group consisting of temperatures from about 16° C. (61° F.) to about 24° C. (75° F.), from about 20° C. (68° F.) to about 23° C. (73° F.), from about 20° C. 68° F.) to about 22° C. (72° F.), and from about 15° C. (59° F.) to about 22° C. (72° F.).

9. The method of claim 1, wherein incubating corneal tissue to a temperature above ambient temperature comprises incubating the corneal tissue at a temperature approximating body temperature or approximating the cornea's natural functional temperature.

10. The method of claim 9, wherein incubating the corneal tissue at a temperature approximating body temperature or approximating the cornea's natural functional temperature comprises incubating the corneal tissue at a temperature in a range selected from the group consisting of temperatures from 25° C. (75° F.) to 38° C. (100° F.), from 30° C. (86° F.) to 38° C. (100° F.), from 31° C. (89° F.) to 37° C. (99° F.), from 32° C. (90° F.) to 36° C. (97° F.), from 33° C. (91° F.) to 36° C. (97° F.), from 33° C. (91° F.) to 35° C. (95° F.), and from 33° C. (91° F.) to 34° C. (93° F.), for at least a 0.5 hour.

11. The method of claim 9, wherein incubating corneal tissue to a temperature above ambient temperature or approximating the cornea's natural functional temperature comprises incubating the corneal tissue to a temperature sufficient to enhance endothelial cell border definition relative to that seen at ambient temperature.

12. The method of claim 8, wherein incubating corneal tissue to a temperature above ambient temperature or approximating the cornea's natural functional temperature comprises incubating the corneal tissue to a temperature in the range of 27° C. (81° F.) to 38° C. (100° F.), 27° C. (81° F.) to 37° C. (99° F.), 27° C. (81° F.) to 36° C. (97° F.), 27° C. (81° F.) to 35° C. (95° F.), and 27° C. (81° F.) to 34° C. (93° F.).

13. The method of claim 1, further comprising validating the corneal tissue for transplantation purposes, based on the assessed corneal tissue quality.

14. The method of claim 1, wherein the hypothermic corneal storage media comprises at least one ingredient selected from the group consisting of: an aqueous nutrient and electrolyte solution; a glycosaminoglycan; a deturgescent agent; an energy source; a buffer system; an antioxidant; a membrane stabilizing component; an antibiotic and/or antimycotic; and ATP precursors, in a combination effective in maintaining corneal deturgescence, thickness, and transparency.

15. The method of claim 1, wherein the hypothermic corneal storage media comprises at least one selected from the group consisting of: Optisol; Optisol GS: Dexsol; Life4C; McCarey-Kaufman (M.K.); and Eusol-C.

16. The method of claim 1, wherein the hypothermic corneal storage media comprises chondroitin sulfate and/or dextran 40.

17. A validated corneal tissue sample validated for transplantation purposes using a method for assessing corneal tissue quality according to claim 1.

18. The corneal tissue sample of claim 17, wherein the corneal tissue comprises a mammalian cornea validated for transfer into a mammalian recipient.

19. The corneal tissue sample of claim 18, wherein the corneal tissue comprises a human cornea validated for transfer into a human recipient.

20. The method of claim 13, further comprising transferring the validated corneal tissue.
